(19) United States
(12) Patent Application Publication
(10) Pub. No.: US 2012/0323138 A1
(43) Pub. Date: Dec. 20, 2012

(54) METHOD AND DEVICE FOR EVOKING TEMPORAL PAIN SUMMATION

(75) Inventors: Lars Arendt-Nielsen, Aalborg (DK); Thomas Graven-Nielsen, Aalborg (DK)

(73) Assignee: AALBORG UNIVERSITET, Aalborg Ø (DK)

(21) Appl. No.: 13/580,542

(22) PCT Filed: Feb. 24, 2011
(86) PCT No.: PCT/DK2011/050058

§ 371 Date: Aug. 22, 2012

(30) Foreign Application Priority Data
Feb. 26, 2010 (DK) PA 2010 70073

(51) Int. Cl. A61B 10/00; A61B 5/05

(52) U.S. Cl. 600/552; 600/557; 600/554; 600/555

(57) ABSTRACT

A device for evoking a temporal summation of pain in a subject. The device comprises a stimulation element (SE) arranged generating a physical stimulation on a skin surface area of at least 20 cm² according to a control signal (CS), where the stimulation (PS) evokes pain in the subject. A processor (PR) generates the control signal (CS) to the stimulation element (SE) so as to provide a repetitive stimulation pattern comprising at least repetitions of: a)applying stimulation for a period (SP) of 0.5-120 seconds, b)stopping stimulation for a period (TP) of 0.5-20 seconds. Preferred periods are 1-3 seconds for both stimulation and intermediate stopping periods. The stimulation element may include an inflatable tourniquet arranged for providing a compressional stimulation of an arm or a leg. However, other types of stimulation elements may be used such as electric, heating, mechanical stimulation or the like may be used. The device is suited for providing a measure of temporal summation in a subject with different intensities, and this measure can be used for determining if the subject suffers from central nervous sensitization which is an important diagnosis with respect to effective analgesic treatment. In some embodiments, the device is arranged to collect pain responses during the stimulation and to calculate a measure of temporal summation of the subject accordingly, e.g. in the form of a rate of pain change (VAS) versus time (T).

### FIELD OF THE INVENTION

The present invention relates to the field of medical methods and devices. More specifically, the invention relates to a method and a device for providing a physical stimulation to a subject to evoke temporal pain summation in the subject. Especially, the method and device are suited for determining pain sensitization of the central nervous system.

### BACKGROUND OF THE INVENTION

Pain and pain sensitization of the central nervous system, i.e. the disorder where the central nervous system is hypersensitive or hyposensitive to pain, plays an important role in a number of diseases with chronic pain involved, e.g. fibromyalgia. For correct analgesic treatment of pain and for screening analgesic compounds it is important to determine if the pain is caused by a local or central phenomenon, since specific medication is vital for effective treatment of pain.

However, reliable methods and devices suited for basic experiments and clinical examination of a subject with respect to pain, pain hyper-sensitization and pain hypo-sensitisation of the central nervous system do not exist.

Application of physical stimulation with the research purpose of temporal summation of pain summation is usually done by applying a physical stimulation on the subject with a hand-held algometer probe with a tip having an area of the order of 1 cm². The experimenter presses the probe tip against the subject's skin, e.g. on the arm or leg, in order to evoke pressure pain. To determine a measure of temporal summation of pain in the subject, repeated stimulations are applied, and simultaneously the subject's pain response is recorded, e.g. using an electronic version of the Visual Analogue Scale (VAS). From the tracking of the subject's pain response versus time, and for different stimulation intensities, it can be determined if the subject suffers from an increased pain sensitivity which may be caused by a pain sensitization or pain hypo-sensitization of the central nervous system.

The use of a manually operated algometers (small probes) has a number of disadvantages, such as very localised structures activated and bias of the experimenter in performing the physical stimulation. Furthermore, using an algometer, there is a risk that skin pain sensitization effects will mask the effect of central nervous sensitization. Thus, altogether, a manual algometer is not suited to screen a subject for central nervous pain sensitization and hypo-sensitization. Alternatively, temporal pain summation has been investigated by evoking a constant pain stimulation using an inflatable tourniquet around the subject's arm or leg (Jespersen A et. al. Pain 131, pages 57-62, 2007). However, a disadvantage by such method is local pain sensitizing and de-sensitizing effects that decrease the chance of detecting central nervous pain sensitization and hypo-sensitization.

### SUMMARY OF THE INVENTION

According to the above, it may be seen as an object of the present invention to provide a device and a method suited for clinical examination of temporal pain summation in a subject, so as to be able to evaluate the subject with respect to central nervous pain sensitization and hypo-sensitization, and thereby enabling effective drug treatment.

The above described object and several other objects are intended to be obtained in a first aspect of the invention by providing a device for evoking a temporal summation of pain in a subject comprising
a stimulation element arranged for receiving a control signal and for generating a physical stimulation on a skin surface area of at least 20 cm² accordingly, wherein the physical stimulation is capable of evoking a painful reaction in the subject, and
a processor arranged to generate the control signal to the stimulation element, wherein the processor is arranged to generate the control signal to provide a repetitive stimulation pattern comprising
a) applying stimulation for a period of 0.5-120 seconds,
b) stopping stimulation for a period of 0.5-120 seconds, and
c) repeating steps a) and b) at least 5 times.

With such device, it is possible to automatically perform an examination of temporal pain summation in a subject and thus eliminate bias from the experimenter applying pain with a hand-held algometer. The stimulation process is preferably controlled by a predetermined algorithm run by the processor, e.g. in the form of a Personal Computer with suitable software, or in the form of a dedicated pre-programmed processor being housed within the stimulation device. Thus, once set up by the experimenter, the stimulation pattern is performed in an automated and controlled manner free from human bias and thresholds and stimulus-response functions can be determined.

In addition, the repetitive stimulation pattern ensures that local skin sensitization and de-sensitizing phenomena hinders effective detection of central nervous pain sensitisizing. The fact that the physical stimulation is being applied to a rather large skin area also significantly reduces the risk of central nervous pain sensitizing being unreliably detected due to masking of other effects related to the pain stimulation area. By merely controlling the function of the processor, it is possible to change both timing and intensity related to the physical stimulation, and thus the device is easy to adapt to different stimulation modalities and to subjects with significantly different pain sensitivity.

Furthermore, the device can be provided by standard low cost components, e.g. the processor being a standard Personal Computer, and the stimulation element being in the form of a motorized inflatable tourniquet. Thus, such device is advantageous for clinical testing for central nervous pain sensitizing, thereby assisting a medical doctor in providing the correct diagnosis and thus guide towards an effective analgesic treatment.

The device is easy to modify to a version with multiple stimulation element each suited for providing different types of physical stimulation and thus such modified device is capable of testing temporal pain summation for different types of pain stimulations, e.g. with stimulation elements arranged for stimulating different parts of the subject's body, and/or stimulation elements arranged for stimulating the same area but using different types of stimulation, e.g. compressional stimulation, electrical stimulation etc. Thus, the device of the invention may comprise several stimulation elements integrated within the same physical entity and therefore apply different types of stimulation to substantially the same area. In addition, the device according to the invention may comprise several stimulation elements which are not integrated. Thus, the device can be extended with multiple stimulation elements allowing simultaneous stimulation (or stimulations shifted in time) of different extremities (e.g. arm and leg). This combination is applicable when assessing specific central mechanisms (e.g. descending inhibitory control) controlling the pain sensitivity. It is of course to be understood that though different stimulations elements are physically separated from each other each of these separated stimulation elements may comprise more than one type of stimulation elements.

Therefore an embodiment relates to a device according to the invention with the proviso that said second stimulation element is not partly integrated with said first stimulation element. It may be advantageously to have more than to stimulation elements. Thus, in an embodiment the invention relates to a device further comprising at least one additional stimulation element (SE3).

Assessment of cuff temporal summation on e.g. one leg simultaneously with pain on the other leg may provide important knowledge on how different brain centres “suppress” pain. Especially these parts are presumed to be affected in patients.

By the term “for a period” in relation to defining the duration of “applying stimulation” and “stopping stimulation” periods, it is to be understood that these periods are when e.g. larger than 90% of a target stimulation intensity is reached, and when less than 10% of the target stimulation intensity is reached, respectively. By “stopping stimulation” is understood that in this period the stimulation intensity is significantly reduced compared to the “applying stimulation” period. However, in some embodiments the “stopping stimulation” period means that the stimulation is not necessarily reduced to zero intensity, but rather to a basis target stimulation intensity level significantly lower than during “applying stimulation”, such as a stimulation intensity level within the range slightly below to slightly above the threshold of pain for that type of physical stimulation. Thus, in practice it will take some time for the stimulation element to provide the intended physical stimulation, when the control signal requires a change from one stimulation intensity level to another stimulation intensity level, however with the above definition, the stated periods are well-defined and clearly related to the actual physical stimulation applied to the subject, rather than being dependent on the temporal response of the stimulation element.

The stimulation element is preferably arranged for stimulating an area of 20-1000 cm², such as 40-500 cm², such as 50-200 cm². A rather large stimulation area serves to suppress local sensitization or adaptation phenomena in the skin.

The device is preferably arranged for applying stimulations for a period of 0.5-120 seconds, such as 1-60 seconds, such as 1-20 seconds, such as 1-10 seconds, such as 1-3 seconds. Preferably, all stimulation periods in a repetitive stimulation pattern have the same length.

The device is preferably arranged for stopping stimulations for a period of 0.5-120 seconds, such as 1-60 seconds, such as 1-20 seconds, such as 1-10 seconds, such as 1-3 seconds. Preferably, all stopping periods in a repetitive stimulation pattern have the same length.

The device is preferably arranged for repeating the stimulation pattern 5-100 times, such as 5-10 times, such as 5-25 times.

The stimulation element may be arranged for providing at least one stimulation selected from the group consisting of mechanical stimulation, compressional stimulation, vibrational stimulation, thermal stimulation such as heating and cooling, electrical stimulation such as an alternating or direct current and radiation. In a further embodiment the device comprises at least two, such as at least three, or such as at least four stimulation elements. Such elements may be integrated as shown in FIG. 3 or be physically separated to allow stimulation of different parts of the body.

Preferably, the stimulation element is arranged for providing the physical stimulation in the form of compressional stimulation, e.g. the stimulation element may be arranged for providing compressional stimulation intensity in the ranges of 3-100 kPa, such as 5-80 kPa, and preferably the stimulation element is arranged to provide the compressional stimulation on a circumferential area of the body, e.g. around a leg or an arm. The stimulation element may be arranged for providing a basis compressional stimulation intensity of 0.5-3 kPa between the compressional stimulations, i.e. in the “stopping stimulation” periods.

In one embodiment, a part of the stimulation element being arranged for contact with the subject is a tourniquet with an inflatable chamber, e.g. in the form of an inflatable tourniquet such as used for blood pressure measurements. Especially, the tourniquet may comprise multiple inflatable chambers arranged for inducing pressure, such as two chambers, such as three chambers, such as more than three chambers, such as each of the multiple inflatable chambers being arranged for providing a substantial evenly distributed pressure on respective skin surface areas. Such additional chambers may be used to provide pain stimulation enabling examination of a subject's spatial pain summation. The stimulation element may comprise a pressure generator arranged to change the applied pressure at a rate of at least 10 kPa per second, such as a rate of at least 40 kPa per second, such as a rate of at least 40 kPa per second, thus allowing a rather rapid increase and decrease in physical stimulation so as to allow stimulation and non-stimulation periods of the order of 1-3 seconds. Especially, such pressure generator may be arranged to actively lowering the pressure, such as by suction at a pressure below the surrounding environmental pressure, thereby speeding up the transition between stimulation and non-stimulation.

Preferably, the stimulation element is arranged for providing stimulation of a body area selected from the group consisting of: part of the upper arm, part of the forearm, part of the thigh, part of the lower leg, the upper part of the head, fingers, hand, and feet.

Especially it may be preferred that a part of the stimulation element arranged for contact with the subject is made of substantially non-metallic material, thereby allowing this part of the stimulation element to be attached to the subject and functional during application of a strong magnetic field, such as during MR scanning which can then be used to monitor brain activity during application of the physical stimulation and thereby serve as a measure of evoked pain.

In some embodiments, the device comprises a second stimulation element arranged for providing a second physical stimulation, such as electrical stimulation, different from compressional stimulation in accordance with a second control signal from the processor. Thus, in such embodiment with multiple stimulation elements, a versatile pain stimulation device is provided which is capable of testing the subject with respect to various pain types, e.g. at different parts of the body. Especially, a part of the second stimulation element arranged for contact with the subject is at least partly integrated with a part of the first stimulation element arranged for contact with the subject, such as an electric stimulation electrode attached on an inside of an inflatable tourniquet. With such combined stimulation element a compact and easy way of testing different parameters relating to pain sensitivity is provided, since only the tourniquet need to be mounted on the subject, and thereafter an automated computer controlled experiment can be performed without involvement of an experimenter.

In addition, the device according to the invention may comprise several stimulation elements which are not integrated. Thus, the device can be extended with multiple stimulation elements allowing simultaneous stimulation of different extremities (i.e. arm and leg). This combination is applicable when assessing specific central mechanisms (e.g. descending inhibitory control) controlling the pain sensitivity. It is of course to be understood that though different stimulations elements are physically separated from each other each of these separated stimulation elements may comprise more than one type of stimulation elements.

Therefore in an embodiment the invention relates to a device according to the invention, with the proviso that said second stimulation element is not partly integrated with said first stimulation element. The number of separated stimulation elements forming part of the device may vary. Thus, in an embodiment the further comprising at least one additional stimulation element (SE3). Thus, according to the invention the device may comprise more than one stimulation element physically separated from the first stimulation elements such as 1-10, such as 2-5 such as 3-5 stimulation element physically separated from said first stimulation element.

In some embodiments, the device comprises means for receiving data on evoked pain in a subject, such as means for receiving data on evoked pain in the form of at least one of: a Visual Analogue Scale, brain signals measured by electrodes on the head of a subject, MRI scanning data, and fMRI scanning data. The device may be built together in one dedicated device, or the processor and the means for receiving data on evoked pain in the subject may be separated into separate units. However, a Personal Computer may perform both functions with a simple user interface, e.g. in the form of a pain response panel connected to the Personal Computer. The device may further comprise calculation means arranged to calculate a measure of temporal summation of pain for the subject, based on the data on evoked pain in the subject, such as a measure of pain increase versus time in response to the stimulation pattern. Especially, the calculation means may be arranged to calculate a measure of spatial summation of pain for the subject.

In a second aspect, the invention provides a method for evoking a response of temporal summation of pain in a subject, the method comprising applying a repetitive stimulation pattern on a skin surface area of at least 20 cm² evoking temporal summation of pain in the subject, wherein said stimulation pattern comprises
applying stimulation for a period of 0.5-120 seconds,
stopping stimulation for a period of 0.5-120 seconds, and
repeating steps a) and b) at least 5 times.

In an embodiment the invention further relates to applying different stimulation intensities in the different stimulation periods. The stimulation intensity of course depends on the type of stimulation as described above. The person skilled in the art would be able to adjust the different parameters. Thus the stimulation may the same within a stimulation pattern or vary between one or more of the stimulation periods within the stimulation pattern.

The same advantages and embodiments as mentioned for the first aspect apply as well for the second aspect.

In a third aspect, the invention provides a method for estimating the effect of a compound or composition on temporal pain summation in a subject comprising
providing the compound or composition to a subject,
measuring the temporal pain summation in said subject using the method according to the second aspect, and
comparing the measured temporal pain summation to a control, standard or recordings before providing the compound.

In a fourth aspect, the invention provides a method for estimating the effect of a treatment on temporal pain summation in a subject comprising
obtaining a first measurement of temporal pain summation in the subject according to the second aspect,
providing a treatment to said subject,
obtaining a second measurement of temporal pain summation in the subject according to the second aspect, and
comparing the two measurements.

Especially, the treatment may be selected from: drug treatment, physiotherapeutic treatment, cognitive treatment, clinical diagnostics, pre- and postoperative monitoring.

The compound or composition may be one of: orally, intravenously, topically, epidurally, intrathecally, nasally, locally, intramuscularly, or subcutaneously applied.

It is appreciated that the same advantages and equivalent embodiments as described for the first aspect apply as well for the second, third, and fourth aspects.

### BRIEF DESCRIPTION OF THE FIGURES

The aspects and embodiments according to the invention will now be described in more detail with regard to the accompanying figures. The figures show one way of implementing the present invention and is not to be construed as being limiting to other possible embodiments falling within the scope of the attached claim set.

FIG. 1 shows schematic diagram of a simple embodiment,

FIG. 2 shows an example of timing of the physical stimulation pattern,

FIG. 3 shows an embodiment with multiple stimulation elements,

FIG. 4 shows an embodiment with a pain response interface and calculating means arranged to output a measure of temporal pain summation of a subject, and

FIG. 5 illustrates a graph with pain response data versus time for a subject with central nervous pain sensitization.

### DETAILED DESCRIPTION

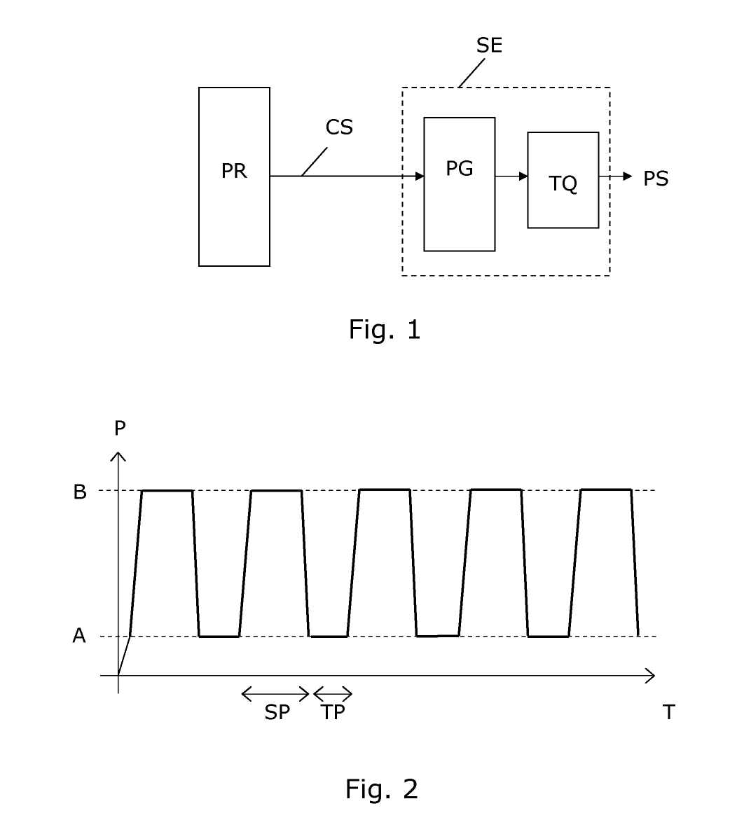

FIG. 1 shows a schematic embodiment of a device for evoking a temporal summation of pain in a subject. A processor PR, e.g. a computer, generates an electric control signal CS that controls the function of a stimulation element SE which generates a physical stimulation PS on the skin of a subject according to the control signal CS. In this embodiment, the stimulation element includes a pressure generator PG which receives the control signal CS and generates air pressure to an inflatable chamber of a tourniquet TQ, e.g. a tourniquet TQ suited to fit around an arm or a leg of the subject. Thus, such stimulation element SE is suited to apply a physical stimulation in the form of compressional stimulation around a part of the subject's body. A tourniquet TQ of the types used for blood pressure measurements can be used, and with a typical size, a skin area of the subject's of the order of 100 cm² or more is applied with the compressional stimulation. The pressure generator PG may be connected to the tourniquet TQ by means of a pipe, thus allowing a motorized pressure generator PG to be placed away from the subject. The pressure generator PG preferably includes an air compressor connected to the tourniquet TQ via a valve arrangement which is controlled in accordance with the control signal CS. Hereby it is possible to inflate and deflate the air chamber of the tourniquet TQ rapidly, so as to allow low transition times between consecutive stimulus and non-stimulus periods of the stimulation pattern. As described previously the tourniquet may comprise more than one inflatable chamber.

FIG. 2 illustrates a graph of a possible stimulation pattern which may be used for the embodiment of FIG. 1 or the principles illustrated in FIG. 2 may as well be used in other embodiments. The bold line indicates the tourniquet pressure P as a measure of intensity of the compressional stimulation applied versus time T. The stimulation period is indicated by SP, i.e. the period where the pressure P has reached a target level B for stimulation, whereas the non-stimulation period is indicated by TP, i.e. the period where the pressure P has reached a target level A for non-stimulation. It is to be understood that any of the values B, SP, TP and A may vary within a stimulation pattern.

The stimulation with pressure P at a level of B is illustrated as repeated 5 times, however depending on the subject and the chosen stimulation level B, the stimulation may be repeated more times. Preferably, both the stimulation period

SP and the non-stimulation period TP are selected with the interval of 1 to 3 seconds, e.g. such that SP=TP. However, SP may be chosen shorter than TP, e.g. SP=1 second and TP=3 seconds.

The stimulation pressure level B depends on the subject and on the part of the body being stimulated. However, preferred values for B are within the interval 3-100 kPa, such as 5-80 kPa.

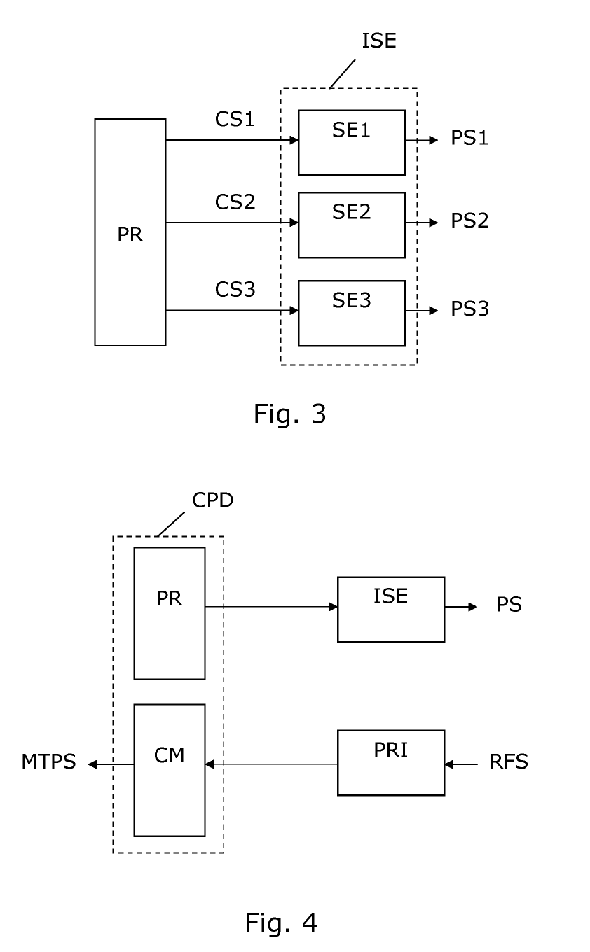

FIG. 3 illustrates an embodiment being a variant of the one illustrated in FIG. 1. The processor PR is arranged to generate three individual control signals CS1, CS2, CS3 to respective stimulation elements SE1, SE2, SE3, and the stimulation elements SE1, SE2, SE3 are arranged to generate respective physical stimuli PS1, PS2, PS3 accordingly. The dashed line indicates that the three stimulation elements SE1, SE2, SE3 are built together as one or several integrated stimulation elements (ISE). Especially, the stimulation elements SE1, SE2, SE3 may be based on different stimulation principles and thus generate different types of physical stimuli PS1, PS2, PS3. E.g. SE1 may be a compressional tourniquet, while SE2 is an electric stimulation electrode arranged on an inside surface of the tourniquet, and finally, SE3 may be an electric heating element also arranged inside the tourniquet. Additional ISEs with similar or other stimulation modalities 30 is optional. In such case, the device is capable of generating a compressional stimulation as PS1, electric chock as PS2, and heat as PS3. It is to be understood that the device according to the invention may comprise more than one integrated stimulation element (ISE) such as 2, such as 3, such as 4, such as 1-10, such as 1-5 or such as 1-3 integrated stimulation elements.

FIG. 4 illustrates an embodiment with a pain response interface PRI arranged to receive a pain response RFS from the subject. A processor PR generates a control signal to a stimulation element ISE, e.g. based on an inflatable tourniquet, and the stimulation element ISE applies a physical stimulation PS accordingly. The pain response RFS is transformed by the interface PRI into a signal which is communicated, wired or wirelessly, to a calculation means CM, e.g. a separate processor or merely a separate algorithm running on the same processor PR that generates the control signal to the stimulation element ISE. The calculation means CM calculates a measure of temporal pain summation in the subject based on the collected pain responses RFS, e.g. in the form of a logging of VAS data during application of the physical stimulation PS which is performed such as already described. Again it is to be understood that the device or system according to the invention may comprise more than one ISE and more than one PRI. Similar it is to be understood that the one or more stimulation elements may not be integrated and therefore only able to provide one type of stimulation.

In the illustrated embodiment, both the processor PR and the calculation means CM are housed within a computer device CPD, e.g. a Personal Computer, or a dedicated stand-alone device. Such embodiment as illustrated in FIG. 4 is suited for assisting a medical doctor in diagnosis regarding treatment of pain, e.g. at a hospital or in a GP consultancy. With the measure of temporal summation of pain of a subject, e.g. in the form of a rate of pain change versus time, as will be described below, it is possible to determine if the subject suffers from central nervous pain sensitization, and thus it is possible to propose an effective analgesic treatment accordingly.

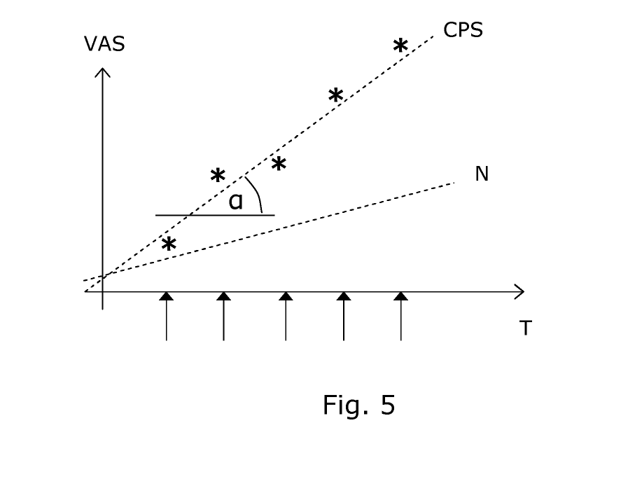

FIG. 5 illustrates an example of a measure of temporal pain summation in the form of a rate of change a of pain response VAS versus time T. The graph in FIG. 5 shows a fictive set of pain response data (asterisks) on a VAS scale from a subject in response to application of a physical stimulation in a repetitive patter, as described above. In FIG. 5 arrows indicate short period, e.g. 1-3 seconds, of physical stimulation with the same predetermined intensity, e.g. compressional stimulation applied by an inflatable tourniquet at an intensity evoking a pain above the threshold of pain, e.g. by providing a pressure of 30 kPa. In the example, a rate of pain increase for a normal subject is indicated by the dashed line N, whereas a linear regression line through the pain response data is sketched as dashed line CPS with a rate of change a of pain response VAS versus time T. The rate of change a of pain response VAS versus time T indicates in this example a significant increase compared to the normal line N, and thus it indicates that the subject suffers from central nervous pain sensitization.

The rate of change a of pain response VAS versus time T may be calculated in calculation means CM of the embodiment shown in FIG. 4 as a simple example of an aid for a medical doctor in diagnosing a subject, e.g. by comparing a for a given subject with a tabulated value for a normal subject.

Similar, when testing a treatment (such as a drug or composition) an effect of the drug on temporal pain summation may be seen as the a for a subject after treatment will approach the a for the subject measured before the treatment. Thus, an effect of a treatment may be measured.

To sum up, the invention provides a device for evoking a temporal summation of pain in a subject. The device comprises a stimulation element SE arranged generating a physical stimulation on a skin surface area of at least 20 cm² according to a control signal CS, where the stimulation PS evokes pain in the subject. A processor PR generates the control signal CS to the stimulation element SE so as to provide a repetitive stimulation pattern comprising at least 5 repetitions of: a) applying stimulation for a period SP of 0.5-120 seconds, b) stopping stimulation for a period TP of 0.5-120 seconds. Preferred periods are 1-3 seconds for both stimulation and intermediate stopping periods. The stimulation element may include an inflatable tourniquet arranged for providing a compressional stimulation of an arm or a leg. However, other types of stimulation elements may be used such as electrical, heating, mechanical stimulation or the like may be used. The device is suited for providing a measure of temporal summation in a subject, and this measure can be used for determining if the subject suffers from central nervous sensitization which is an important diagnosis with respect to effective analgesic treatment. In some embodiments, the device is arranged to collect pain responses during the stimulation and to calculate a measure of temporal summation of the subject accordingly, e.g. in the form of a rate of change a of pain VAS versus time T.

Although the present invention has been described in connection with the specified embodiments, it should not be construed as being in any way limited to the presented examples. The scope of the present invention is to be interpreted in the light of the accompanying claim set. In the context of the claims, the terms “including”, “includes”, “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A device for evoking a temporal summation of pain in a subject comprising:
a stimulation element (SE) arranged for receiving a control signal (CS) and for generating a physical stimulation on a skin surface area of at least 20 cm² accordingly, wherein the physical stimulation (PS) is capable of evoking a painful reaction in the subject, and
a processor (PR) arranged to generate the control signal (CS) to the stimulation element (SE), wherein the processor (PR) is arranged to generate the control signal (CS) to provide a repetitive stimulation pattern comprising
a) applying stimulation for a period (SP) of 0.5-120 seconds,
b) stopping stimulation for a period (TP) of 0.5-120 seconds, and
c) repeating steps a) and b) at least 5 times.

2-31. (canceled)

32. The device according to claim 1, wherein said device is further adapted for applying different stimulation intensities.

33. The device according to claim 1, wherein the stimulation element (SE) is arranged for stimulating an area of 20-1000 cm².

34. The device according to claim 1, arranged for applying stimulations for a period (SP) of 0.5-120 seconds.

35. The device according to claim 1, arranged for stopping stimulations for a period (TP) of 0.5-120 seconds.

36. The device according to claim 1, arranged for repeating the stimulation pattern 5-100 times.

37. The device according to claim 1, wherein the stimulation element (SE) is arranged for providing at least one stimulation (PS) selected from the group consisting of compressional stimulation, mechanical stimulation, vibrational stimulation, thermal stimulation, electrical stimulation, and radiation.

38. The device according to claim 1, wherein the stimulation element (SE) is arranged for providing the physical stimulation (PS) in the form of compressional stimulation.

39. The device according to claim 38, wherein a part of the stimulation element (SE) is arranged for contact with the subject, is a tourniquet (TQ) with an inflatable chamber.

40. The device according to claim 38, wherein the stimulation element (SE) comprises a pressure generator (PG) arranged to change the applied pressure at a rate of at least 10 kPa per second.

41. The device according to claim 40, wherein the pressure generator (PG) is arranged to actively lower the pressure.

42. The device according to claim 1, wherein the stimulation element (SE) is arranged for providing stimulation of a body area selected from the group consisting of part of the upper arm, part of the forearm, part of the thigh, part of the lower leg, the upper part of the head, fingers, hand, and feet.

43. The device according to claim 37, further comprising a second stimulation element (SE2) arranged for providing a second physical stimulation (PS2) different from compressional stimulation in accordance with a second control signal (CS2) from the processor (PR).

44. The device according to claim 43, wherein a part of the second stimulation element (SE2) arranged for contact with the subject is at least partly integrated with a part of the first stimulation element (SE1) arranged for contact with the subject.

45. The device according to claim 1, further comprising a means (PRI) for receiving data on evoked pain in a subject.

46. The device according to claim 45, further comprising a calculation means (CM) arranged to calculate a measure of temporal summation of pain (MTSP) for the subject, based on the data on evoked pain in the subject (VAS).

47. The device according to claim 46, wherein the calculation means (CM) is further arranged to calculate a measure of spatial summation of pain for the subject.

48. A method for evoking a response of temporal summation of pain in a subject, the method comprising applying a repetitive stimulation pattern on a skin surface area of at least 20 cm² evoking temporal summation of pain in the subject, wherein said stimulation pattern comprises:
applying stimulation for a period of 0.5-120 seconds,
stopping stimulation for a period of 0.5-120 seconds, and
repeating steps a) and b) at least 5 times.

49. A method for estimating the effect of a compound or composition on temporal pain summation in a subject comprising:
providing the compound or composition to a subject,
measuring the temporal pain summation in said subject using the method according to claim 48, and
comparing the measured temporal pain summation to a control, standard or recordings before providing the compound or composition.

50. A method for estimating the effect of a treatment on temporal pain summation in a subject comprising:
obtaining a first measurement of temporal pain summation in the subject according to claim 48,
providing a treatment to said subject,
obtaining a second measurement of temporal pain summation in the subject according to claim 48, and
comparing the two measurements.
